(19) United States
(12) Patent Application Publication
(10) Pub. No.: US 2025/0301978 A1
(43) Pub. Date: Oct. 2, 2025

(54) MULTIPURPOSE AUTONOMOUS POLLINATION AND FERTIGATION SYSTEM FOR A CONTROLLED GROWING ENVIRONMENT

(71) Applicants: Habiba Bougherara, Toronto (CA); Ahmed Sarwar, Toronto (CA); Lesley Campbell, Toronto (CA); Serena Sbrizzi, Toronto (CA)

(72) Inventors: Habiba Bougherara, Toronto (CA); Ahmed Sarwar, Toronto (CA); Lesley Campbell, Toronto (CA); Serena Sbrizzi, Toronto (CA)

(21) Appl. No.: 19/060,306

(22) Filed: Feb. 21, 2025

Related U.S. Application Data
(60) Provisional application No. 63/569,763, filed on Mar. 26, 2024.

(51) Int. Cl. A01H 1/02; A01G 9/24

(52) U.S. Cl. CPC A01H 1/027; A01G 9/24

(57) ABSTRACT

A method for pollinating a plant in an indoor growing environment, said plant having a top, a bottom and at least one anther with exposed pollen, the method including: i. applying positive air pressure on one side of the plant; ii. applying negative air pressure on another side of the plant; whereby the negative air pressure draws air supplied by the positive air pressure on the one side of the plant from the other side of the plant past the bottom of the plant generating an air vortex releasing the pollen from the anther and circulating the pollen to a stigma in a vicinity proximal and distal the plant resulting in pollination.

### CROSS-REFERENCE TO RELATED APPLICATIONS

The present application claims the benefit of U.S. provisional Patent Application No. 63/569,763 entitled “Multipurpose Autonomous Pollination and Fertigation System for a Controlled Growing Environment” filed Mar. 26, 2024, the entire contents of which are incorporated herein by reference in their entirety.

### FIELD OF THE DISCLOSURE

This disclosure relates to a multipurpose autonomous pollination, fertigation and misting system for a controlled growing environment and methods related thereto.

### BACKGROUND

Some plants reproduce via pollination, when genetic material is transferred amongst plants, flowers of plants, or within a flower. Pollination may be brought about by auto-pollination where the stigma and anther of a plant make contact with each other without outside intervention, by manually transporting pollen to a stigma, by transporting pollen from anther to stigma via controlled movement of abiotic factors like wind or water, or by raising insects that pollinate plants.

At present, to produce crops that result in fruit or seed production (either because that is the product harvested or to create the next generation), many crops require pollination, either to create fruit and/or seeds or to improve the amount of seed or fruit production.

Protected or controlled environment agriculture encompasses a wide variety of modern horticultural environments, including but not limited to high tunnels, low tunnels, greenhouses, and controlled agriculture (including vertical farming). These horticultural methods are of growing interest, as they are recognized tools for increasing food security and sustainable farming (Avgoustaki, D. D., & Xydis, G. (2020). Chapter 1. How energy innovation in indoor vertical farming can improve food security, sustainability, and food safety?. In Advances in Food Security and Sustainability (Vol. 5, pp. 1-51). Elsevier; Banerjee, S., & Punekar, R. M. (2020). A sustainability-oriented design approach for agricultural machinery and its associated service ecosystem development. Journal of Cleaner Production, 264, 121642). Although high tunnels and greenhouses offer benefits such as an extended growing season (Kang, Y., Chang, Y. C. A., Choi, H. S., & Gu, M. (2013). Current and future status of protected cultivation techniques in Asia. Acta Hortic, 987, 33-40; Huff, P. (2015). Extending the growing season. Institute for Agriculture and Trade Policy), and protection from detrimental weather conditions (Lamont, W. J. 2005. Overview of the Use of High Tunnels Worldwide. Hort Technology 19(1), 25-29; Ponce et al., 2004), some limitations still remain (Beshada E, Zhang Q & Boris R (2006). Winter performance of a solar energy greenhouse in southern Manitoba. Canadian Biosystems Engineering 48(5): 1-8; Dorais, M., & Gosselin, A. (2000). Physiological response of greenhouse vegetable crops to supplemental lighting. In IV International ISHS Symposium on Artificial Lighting, 580 (November), 59-67) including that pollination may be reduced although inconsistently so (Seez, A., Morales, C. L., Ramos, L. Y., & Aizen, M. A. (2014). Extremely frequent bee visits increase pollen deposition but reduce drupelet set in raspberry. Journal of Applied Ecology, 51(6), 1603-1612).

Many controlled environment agriculture crop operations employ artificial pollination tools (Caldeira, K. G., Chan, A. K., Hyde, R. A., Kare, J. T., MAnkin, M. N., Pan, T. S., & Wood, L. L. (2015). Systems and methods for selective pollination (Patent No. US20160353661A1); Chun, W., Zhihong, W., Mingjin, L., Yuewei, S., & Xiaobing, X. (2014). Artificial pollination pen for crops (Patent No. CN203872734U); Holcroft, D. M., and P. Allan. 1994. Artificial pollination of kiwifruit. Journal of the Southern African Society for Horticultural Sciences 4, 21-23; Hongyun, H. (2013). Non-contact artificial pollinator (Patent No. CN203692122U); Zhang, Y., & Li, H. (2013). Crop natural pollination greenhouse (Patent No. CN203353293U).) for plants with particular pollination requirements, but the labour involved to cause artificial pollination with many of the tools currently available at scale is large, and pollination is easily heterogeneous, affecting the shape and the quality of fruit produced, and reducing economic opportunities for farmers.

Anemophilous (wind-borne) pollination is common when insect pollinators are absent but if a plant requires cross-pollination in order to produce increased fruit set, then the weak wind movement in a greenhouse or controlled facility will likely limit fruit set. Weak wind movement only moves very light pollen very small distances and as a result tends to result in self-pollination (between flowers within a single plant), not cross-pollination (between plants) which limits seed or fruit production.

Current controlled environment agriculture of many plants is limited, in part, due to the pollination requirements. For instance, Aggregate fruits need full cross-pollination of all drupelets to be fully formed (Kronenberg, H. G. (1959). Poor fruit setting in strawberries. I: Causes of a poor fruit set in strawberries in general. Euphytica, 8, 47-57; Whitney, G. G. (1984). The reproductive biology of raspberries and plant-pollinator community structure. American Journal of Botany, 71(7), 887-894. Raspberry (and cane crops more generally) fruits are 30% larger when visited by a pollen dispersal agent, and therefore are generally considered pollinator dependent (Cane, J. H. (2005). Pollination potential of the bee Osmia aglaia for cultivated red raspberries and blackberries (Rubus: Rosaceae). HortScience, 40(6), 1705-1708). However, too much pollinator visitation can actually decrease the number of drupelets in a raspberry plant (Seez, A., Morales, C. L., Ramos, L. Y., & Aizen, M. A. (2014). Extremely frequent bee visits increase pollen deposition but reduce drupelet set in raspberry. Journal of Applied Ecology, 51(6), 1603-1612), therefore careful consideration of pollination frequency must be made, as it will influence the yield of indoor grown Rubus species. Bumblebees are a significant, though not exclusive, pollinator of raspberries (Bataw, A. A. (1996). Pollination ecology of cultivated and wild raspberry (Rubus idaeus) and the behaviour of visiting insects. University of St. Andrews (United Kingdom)) and exhibit a particular mechanism of pollination called buzz pollination where flowers are vibrated by the bumblebee to transfer pollen both from the bumblebee to the flower, and from the flower to the bumblebee, as well as from the flower to itself (Buchmann, S. L. (1983). Buzz pollination in angiosperms. In C. E. Jones and R. J. Little (Eds.). Handbook of experimental pollination ecology, pp. 73-113. Van Nostrand Reinhold, New York). Although commercial raspberry plants are self-fertile (Keep, E. (1968). Incompatibility in Rubus with special reference to R. idaeus L. Canadian Journal of Genetics and Cytology, 10(2), 253-262), self-pollination is limited due to the arrangement of the anthers relative to the stigmas in the flower (Free, J. B. 1993. Insect pollination of crops, 2nd ed. Academic Press, London, U.K). Only the outermost stigmas contact the anthers (Free, J. B. 1993. Insect pollination of crops, 2nd ed. Academic Press, London, U.K) and when self-pollination occurs, it is only documented within the inner 1-4 rows of pistils, which creates fruits with only a few drupelets, resulting in an unsaleable product (Shanks, C. H. (1969). Pollination of raspberries by honeybees, Journal of Apicultural Research, 8(1), 19-21). Previous studies have claimed that agitation by wind or shaking of the plants provided no benefit to pollination (Shanks, C. H. (1969). Pollination of raspberries by honeybees, Journal of Apicultural Research, 8(1), 19-21). Thus, successful, high rates >50% of pollen deposition on stigmas is currently a major barrier to the success of this industry and we have developed an abiotic pollination tool to replicate the insect pollination that is so crucial for maximum fruit yield in many raspberry cultivars.

Pollen grains are released from anthers at about 55-60% moisture content, and that subsequent desiccation is a function of air temperature, relative humidity (RH) and time (Fonseca, A. E., & Westgate, M. E. (2005). Relationship between desiccation and viability of maize pollen. Field crops research, 94(2-3), 114-125.

The current array of artificial pollination tools have numerous drawbacks for application both specifically for aggregate fruits and more broadly for the industry.

First, many artificial pollination tools rely on the collection of pollen prior to pollen dispersal (many patents require pollen to be collected from male plants or stamens on cosexual plants) (Atkinson, D. T., & Atkinson, D. L. (1987). Apparatus for effecting or improving pollination of plants (Patent No. U.S. Pat. No. 4,922,651A). DFC New Zealand Ltd; Hongyun, H. (2013). Non-contact artificial pollinator (Patent No. CN203692122U); Chun, W., Zhihong, W., Mingjin, L., Yuewei, S., & Xiaobing, X. (2014). Artificial pollination pen for crops (Patent No. CN203872734U); Xiannan, H., Tingxiao, T., Liujun, Z., Xialing, Y., Xiaojuan, C., & Yan, L. (2013). Plant pollination robot (Patent No. CN203167757U)).

Second, many artificial pollination tools rely on extensive human labour (Chun, W., Zhihong, W., Mingjin, L., Yuewei, S., & Xiaobing, X. (2014). Artificial pollination pen for crops (Patent No. CN203872734U) Hongyun, H. (2013). Non-contact artificial pollinator (Patent No. CN203692122U)).

Third, when pollen is collected, it is not dispersed to stigmas as soon as anthesis occurs, which results in reduced pollen viability prior to pollen dispersal; this may also result in pollen arriving on cosexual plants when stigma receptivity is declining (Pawar, N., Thakur, N., Negi, M., & Paliwal, A. (2017). Studies on pollen germination, pollination and fruit set in raspberry (Rubus ellipticus) under hilly conditions of Uttarakhand. Int. J. Curr. Microbiol. App. Sci, 6(9), 3698-3703; Hemavati et al., 2019; Dale, A. (1977). Some consequences of pollen storage in the raspberry (Rubus idaeus L.). Euphytica, 26(3), 745-748. Some consequences of pollen storage in the raspberry (Rubus idaeus L.). Euphytica, 26(3), 745-748, Hiregoudar, H., Manju, N. P., & Bundela, M. K. (2019). Studies on pollen quality and quantity, stigma receptivity, pollination and fruit set in raspberry (Rubus paniculatus S.) wild species of Garhwal Himalaya, Uttarakhand, India. International journal of chemical studies, 7, 2211-2216). Accordingly, there is a need for a system that disperses pollen to stigmas as anthesis occurs.

Fourth, when systems are reliant on insects for pollination, the growth facility must also create an environment which supports insect life and foraging (Free, J. B. 1993. Insect pollination of crops, 2nd ed. Academic Press, London, U.K; Kang, Y., Chang, Y. C. A., Choi, H. S., & Gu, M. (2013). Current and future status of protected cultivation techniques in Asia. Acta Hortic, 987, 33-40; Martin-Closas, L., Puigdomènech, P., & Pelacho, A. M. (2006). Pollination Techniques for the improvement of greenhouse tomato production in two crop cycles. In XXVII International Horticultural Congress-IHC2006: International Symposium on Advances in Environmental Control, Automation 761 (August), pp. 327-332; McCartney, L., & Lefsrud, M. (2018). Protected agriculture in extreme environments: a review of controlled environment agriculture in tropical, arid, polar, and urban locations. Applied Engineering in Agriculture, 34(2), 455-473). Moreover, introduction of insects could potentially carry disease into the growing space (Zhang, Y., & Li, H. (2013). Crop natural pollination greenhouse (Patent No. CN203353293U)).

Some designs replace human labour with a robot (Xiannan, H., Tingxiao, T., Liujun, Z., Xialing, Y., Xiaojuan, C., & Yan, L. (2013). Plant pollination robot (Patent No. CN203167757U); Caldeira, K. G., Chan, A. K., Hyde, R. A., Kare, J. T., MAnkin, M. N., Pan, T. S., & Wood, L. L. (2015). Systems and methods for selective pollination (Patent No. US20160353661A1)), however, a robot will take up a significant amount of space, must be accommodated into the design of the grow shelves, and has complex design and is generally extremely expensive (Safreno, D. (2015). Vision-based pollination system (Patent No. US20170042102A1)).

Moreover, all of these pollination systems function for a single purpose, that is, to disperse pollen. Air-borne foliar fertigation is generally used to supplement root-based fertilizers and also relies on dispersing particles through a plant leaf canopy.

### SUMMARY

According to at least one aspect, there is provided a method for pollinating, in one alternative cross-pollinating, in another alternative self-pollinating, at least one plant, preferably at least two plants in a controlled growing environment, said plant having a top, a bottom and at least one anther with exposed pollen, the method comprising: a positive air flow pressure from a top end of said growing environment towards the top of said at least one plant along a first side of said at least one plant towards said bottom of said at least one plant, preferably said at least two plants wherein said first side of said at least one plant is proximate an outside perimeter said growing environment; and a negative air flow pressure resulting in drawing air flow from said bottom of said at least one plant upwards along a second side of said at least one plant towards said top of said at least one plant towards said top end of said growing environment, wherein said second side of said at least one plant is distant said outside perimeter of said growing environment; whereby the positive air flow pressure and the negative air flow pressure generate a turbulent air flow vortex, preferably a plurality of turbulent air flow vortices, more preferably dual/dipolar clockwise vortices, creating i) an air curtain along the outside perimeter of said growing environment and ii) promoting releasing and mixing of the pollen from the at least one anther with exposed pollen and iii) circulating the pollen from the at least one anther with exposed pollen to at least one stigma of the at least one plant, in a vicinity proximal and distal the at least one plant, resulting in pollination.

In at least one alternative, when there are at least two plants, said at least two plants are spaced apart from each other and said positive air flow pressure is provided via a nozzle wherein said nozzle is situated between said at least two spaced apart plants.

In at least one alternative the method further comprises controlling temperature and moisture of the air supplied by the positive air flow pressure.

In at least one alternative the method further comprises fertilizing the at least one plant through the positive air flow pressure.

In at least one alternative the method further comprises misting the plant through the positive air flow pressure.

In at least one alternative the method further comprises fertigating the plant through the positive air flow pressure.

In at least one alternative the method further comprises collecting pollen released from the at least one anther by the negative air flow pressure.

In at least one alternative the method further comprises introducing extraneous pollen to the growing environment.

In at least one alternative the method further comprises providing light to the growing environment, wherein heat from said light is contained by the negative air flow pressure.

In at least one alternative the method further comprises a plurality of plants.

According to at least one other aspect, there is provided a pollination system for a growing environment, in one alternative, and indoor growing environment comprising: at least one support; a top, a bottom, a front, a back, a top central portion and two sides; at least one plant, preferably at least two plants, most preferably a plurality of plants, said at least one plant having a top and a bottom and at least one anther with exposed pollen; said at least one plant supported by the at least one support; at least one air source proximate a side of said top of said growing environment, for positive air flow pressure from the top of said at least one plant along a first side of said at least one plant towards said bottom of said at least one plant, wherein said at least one air source is offset from a center of said at least one plant; at least one air source return said top central portion of said growing environment applying negative air flow pressure from the bottom of said at least one plant along a second side of said at least one plant towards said top of said at least one plant, wherein said second side of said at least one plant is distant said outside perimeter of said growing environment; whereby the positive air flow pressure and the negative air flow pressure generate a turbulent air flow vortex, preferably a plurality of turbulent air flow vortices, creating i) an air curtain along the outside perimeter of said growing environment and ii) promoting releasing and mixing of the pollen from the at least one anther with exposed pollen and iii) circulating the pollen from the at least one anther with exposed pollen to at least one stigma of the at least one plant, in a vicinity proximal and distal the at least one plant, resulting in pollination; at least one light source integrated with said at least one air source return.

In at least one alternative, when there are at least two plants, said at least two plants are spaced apart from each other and said positive air flow pressure is provided via a nozzle wherein said nozzle is situated between said at least two spaced apart plants.

In at least one alternative the pollination system further comprises at least one of a: fertilizer source; a misting source; a fertigation source; and/or combinations thereof.

In at least one alternative the pollination system further comprises a pollen collector.

In at least one alternative said pollen collector is said at least one air source return applying negative air pressure on said ventral side of said at least one plant.

In at least one alternative the pollination system further comprises an extraneous pollination source.

In at least one alternative said extraneous pollination is delivered by said at least one air source applying positive air flow pressure along said first side of said at least one plant.

In at least one alternative the at least one air source further comprises a temperature and humidity controller.

In at least one alternative the at least one air source further comprises a nozzle adjustable in length.

In at least one alternative the nozzle adjustable in length is adjustable in direction.

In at least one alternative the nozzle adjustable in length is adjustable in flow rate.

In at least one alternative the at least one air source is positioned about 365 centimetres (cm) above said top of said at least one plant, preferably between 0 and 120 cm above said top of said at least one plant, wherein said positive air flow pressure has an air flow speed from the nozzles between about 1 meter/second (m/s) to 0.3 m/s respectively.

In at least one alternative, said at lest one air source is positioned about 365 centimetres (cm) proximate said at least one plant, preferably between 0 and 120 cm proximate said at least one plant, more preferably between 1 to 200 cm proximate said at least one plant, wherein said positive air flow pressure has an air flow speed from the nozzles between about 1 meter/second (m/s) to 0.3 m/s respectively.

In at least one alternative said adjustable nozzle further adjusts the direction and flow rate of said at least one air source.

In at least one alternative the at least one air source return is positioned 365 centimetres above said top of said plant, preferably between 0-120 centimetres above said top of said at least one plant, more preferably between 1 to 200 cm above said top of said at least one plant, when air speed from the nozzles is operating at between 1 m/s to 0.3 m/s respectively. In at least one alternative the at least one air source is offset to said first side of said at least one plant.

In one alternative the at least one air source is offset to said first side of said at least one plant.

In at least one alternative, the at least one air source return is centrally located on said top central portion.

In at least one alternative, the at least one air source is located proximate said two sides and distant said at least one air source return.

In at least one alternative the pollen collector recirculates the collected pollen to the at least one air source for positive air flow pressure recirculating the collected pollen to the indoor growing environment.

In at least one alternative, the at least one air source return collects fertigant and recirculates the collected fertigant to the at least one air source for positive air flow pressure recirculating the collected fertigant to the indoor growing environment.

In at least one alternative, the at least one air source return collects moisture in the air and recirculates the collected moisture in the air to the at lest one air source for positive air pressure recirculating the collected moisture in the air to the indoor growing environment.

In at least one alternative the rate of collected pollen, collected fertigant and collected moisture in the air is controlled by said adjustable nozzle of the at least one air source.

In at least one alternative, said plant is a plurality of plants. In one alternative, said plurality of plants are spaced apart from each other.

In at least one alternative, said method and system create at least one air trajectory, preferably a plurality of air trajectories, more preferably a plurality of turbulent air trajectories on both abaxial and adaxial sides of a plant to release pollen from an anther of said plant.

In at least one alternative, said method and system create at least one air trajectory, preferably a plurality of air trajectories, more preferably a plurality of turbulent air trajectories on the bottom and lateral sides of a plant canopy to supply foliar fertigation chemicals and/or foliar plant growth stimulants.

In at least one alternative, said method and system create at least one air trajectory, preferably a plurality of air trajectories, more preferably a plurality of turbulent air trajectories on the bottom and lateral sides of a plant canopy extracting excess or inviable pollen an/or excess foliar particulate chemicals.

In at least one alternative, said at least one air trajectory, preferably a plurality of air trajectories, more preferably a plurality of turbulent air trajectories remain consistent below the plant canopy but vary within the plant canopy facilitating pollen and/or foliar fertigant movement across a growing area of said plant, preferably across a growing area of said plurality of plants.

In at least one alternative, heat for the growing environment is from natural light, artificial light, heat exchanger and combinations thereof.

In at least one alternative, pollen is released into the growing environment from plants within the growing environment and/or from an external pollen source.

In at least one alternative, pollen is released into the growing environment from plants within the growing environment and/or from an external pollen source within the at least one air source.

In at least one alternative, fertigants and growth stimulants are released into the growing environment, preferably from the at least one air source.

In at least one alternative, fertigants and growth stimulants are released into the growing environment from a source within the at least one air source.

In at least one alternative, the at least one air trajectory, preferably a plurality of air trajectories, more preferably a plurality of turbulent air trajectories, is generated by a mechanical device of a ventilation system and controlling air currents directed at the abaxial side of the plant canopy.

In at least one alternative, the at least one air trajectory, preferably the plurality of air trajectories, more preferably a plurality of turbulent air trajectories, comprises air generated by the mechanical device, wherein said air is cleaned of particulate matter (filter or static charge) prior to release in the plant canopy.

In at least one alternative, air for the air source of the growing environment may be ambient air or compressed air.

In at least one alternative said compressed air is compressed carbon dioxide.

In at least one alternative, air is recycled via a return of the ventilation system.

In at least one alternative, there is provided an air distribution and heat extraction system for plant cultivation of a controlled indoor growing environment, said air distribution and heat extraction system comprising: a platform; at least two spaced apart plants on said platform; each of said at least two spaced apart plants comprising at least one of a flower forming multiple inflorescences, at least one leaf, and combinations thereof; at least one air supply outlet generating at least one air trajectory on an abaxial side of the at least one flower or the at least one leaf; at least one air recovery outlet recycling said at least one air trajectory; and at least one light source; wherein the at least one trajectory on the abaxial side of the at least one flower or the at least one leaf moves pollen and/or at least one particulate growth stimulant: a) between inflorescences; b) onto the at least one leaf, and combinations thereof.

In one alternative, the air supply outlet comprises at least one bleed, preferably a plurality of bleeds, along a length of the outlet, dislodging pollen from anthers and creating turbulent air flow for pollen dispersion.

In one alternative, the air supply is length adjustable.

In one alternative, the air supply is a plurality of air supplies being length adjustable and individually controllable.

In one alternative, said methods and systems result in pollen deposition on stigmas of at least 50%, in one alternative between 50 to about 80% pollen deposition on stigmas, in another alternative greater than 80% pollen deposition on stigmas.

In one alternative, said methods and systems allow for pollen dispersion to stigmas at the time of anthesis.

### BRIEF DESCRIPTION OF THE FIGURES

FIG. 1 is a side view of a pollination system, according to one alternative.

FIG. 2 is an end view of the pollination system of FIG. 1.

FIG. 3 is a bottom looking up view of the top wall of the pollination system of FIG. 1.

FIG. 4a is a perspective view of the pollination system of FIG. 1.

FIG. 4b is a perspective view of a pollination system according to one alternative depicting two trays one atop the other.

FIG. 4c is a perspective view of a pollination system according to yet another alternative depicting two adjacent tray system one atop the other.

FIG. 5 depicts two spaced apart plants wherein pollen is being released from an anther of one plant and delivered to a stigma of the same plant and also delivered to another plant and pollen paths in the pollination system.

FIG. 6 depict pollen and fertigants being introduced into the pollination system and pollen being removed from the pollination system.

FIG. 7a depicts an telescopic nozzle according to one alternative.

FIG. 7b depicts a diverging adjustable nozzle according to one alternative.

FIG. 7c depicts a directional nozzle according to one alternative.

FIGS. 8a and 8b depict temperature distribution inside the growing environment.

### DETAILED DESCRIPTION

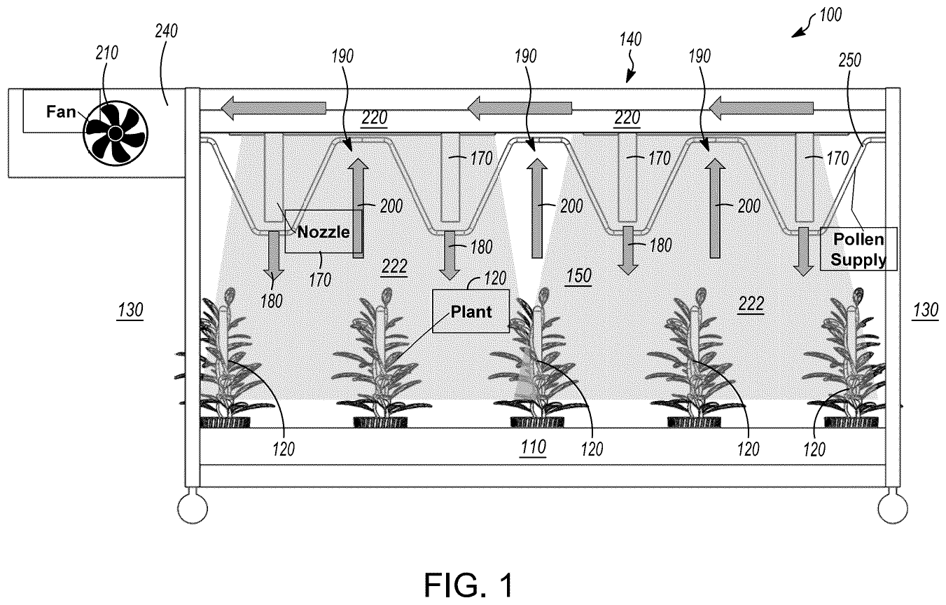

Referring now to FIG. 1, there is depicted a side view of the pollination system 100. The pollination system 100 includes a platform 110, for holding a plurality of plants 120, spaced apart from each other. The pollination system 100 includes sides 130, a top wall 140, a rear 150, and a front wall (not shown) all enclosing the pollination system 100. A plurality of positive pressure air sources, in this alternative elongated nozzles 170, extend downward from the top wall 140 providing positive pressure air stream 180 in a downwardly direction along a side of a plant and between the plurality of plants 120. A plurality of negative air pressure air source returns 190 proximate the top wall 140, drawing the air supplied by the positive air pressure 180 upwards and generating an air vortex upwards 200 releasing the pollen from the anther and circulating the pollen to a stigma in a vicinity proximal and distal the plant resulting in pollination. The negative air pressure air source returns 190 is recirculated by the assistance of a fan 210 keeping the air circulating in the pollination system 100. Any required heat may be introduced into the pollination system 100 by the fan 210 and delivered by the elongated nozzles 170. The pollination system may also comprise a temperature and humidity controller 241 to control the temperature and humidity of the pollination system 100.

The pollination system 100 further includes extraneous pollen supply line 250 running proximate an end of the positive air source nozzles 170 proximate plants 120, wherein the positive air source will assist in the release of extraneous pollen from the extraneous pollen supply line 250. Extraneous pollen supply line 250 having an aperture proximate the end of the positive air source nozzles 170 proximate plants 120 for the release of extraneous pollen into the pollination system 100.

The pollination system 100 further includes a light source 220 integrated with the air pressure air source returns 190. In this alternative the light source is a light emitting diode (LED) source integrated with the air pressure air source returns 190 keeping any unwanted heat from the pollination system 100. Light source 220 provides light to the pollination system. One alternative light beam 222 is shown providing light to the plants 120.

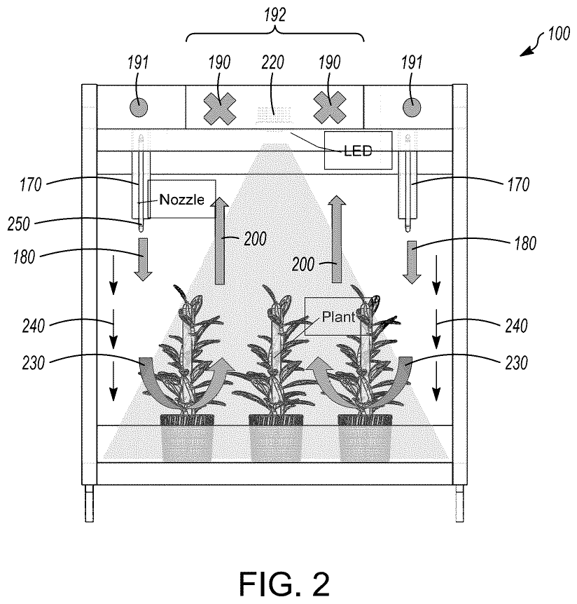

Referring now to FIG. 2, there is depicted an end view of the pollination system of FIG. 1 depicting the negative air pressure air source returns 190, the LED light source 220, the positive pressure air sources 191 and elongated nozzles 170 with positive air stream 180, air vortices upward streams 200 and bottom situated stream 230. Air streams 180, 230 and 200 create an air curtain 240 along the front and rear walls of the pollination system 100. Air curtain 240 mitigates any negative impact from outside the pollination system 100.

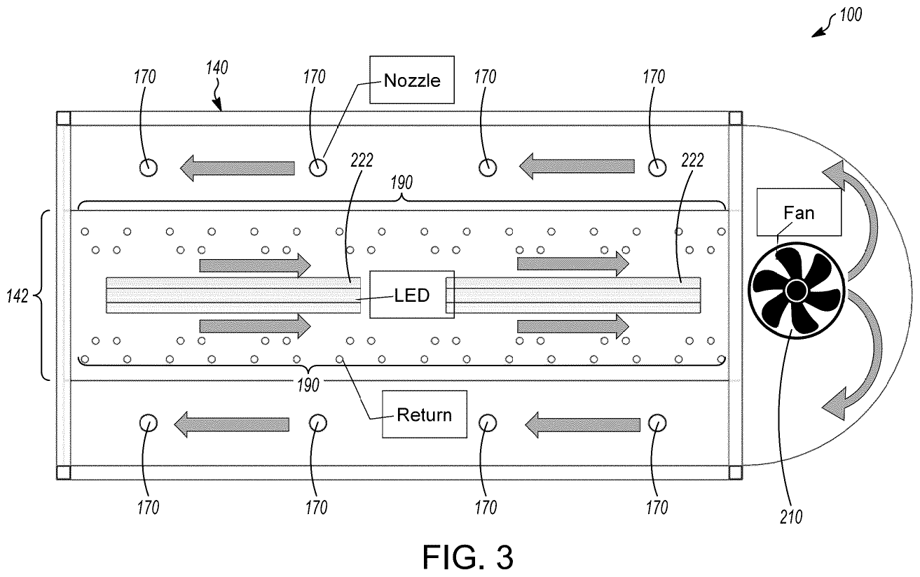

Referring now to FIG. 3, there is depicted a bottom upward view of the pollination system 100 depicting one alternative of the fan 210 being the source of the positive pressure air and negative pressure air, and an arrangement of the elongated nozzles 170 providing the positive pressure air, being situated along the perimeter side of the top wall 140 of the pollination system, and the negative air pressure source returns 190, here a plurality of spaced apart aperture throughout the top wall 140, being situated along the central area 142 of the top wall 140 saddled at each side by the elongated nozzles 170. The LED light source 222 being centrally situated running a length of the central area 142. In this alternative, the LED light sources 222 runs along the length of the central area 142 and parallel to the negative air pressure source returns 190 facilitating humidity and temperature management as well as maintaining pollen viability in the pollination system 100. In this alternative, the LED light source 222 is contained within an air return enclosed space 192 facilitating the management of heat within the pollination system 100 and extracting unwanted heat generated by the LED light source 222 and mitigating unwanted heat into the growing space of the pollination system 100. Another benefit of this arrangement mitigates damage to any pollen by unwanted heat. Although a fan 210 is depicted herein, the source of positive and negative air pressure may be an existing heating, ventilation and air conditioning (HVAC) system or the like.

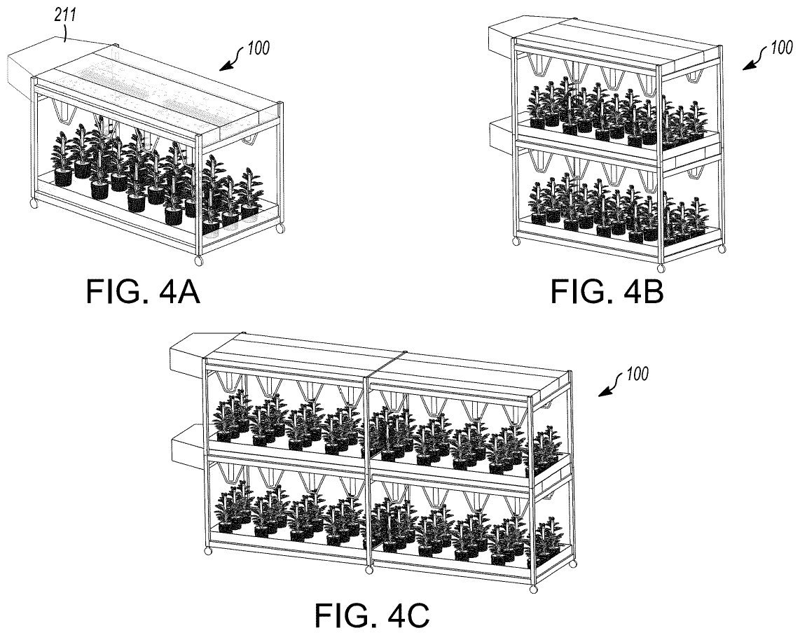

Referring now to FIG. 4a, there is depicted a perspective view of the pollination system 100 as described in FIGS. 1-3 depicting the pollination system 100 with fan housing 211 housing fan 210 (not seen).

Referring now to FIG. 4b, there is depicted a perspective view of an alternative of the pollination system 100 one atop the other.

Referring now to FIG. 4c, there is depicted a perspective view of an alternative of the pollination system 100 with one adjacent the other and one atop the other. This depicts the modularity and expandability aspect of the system.

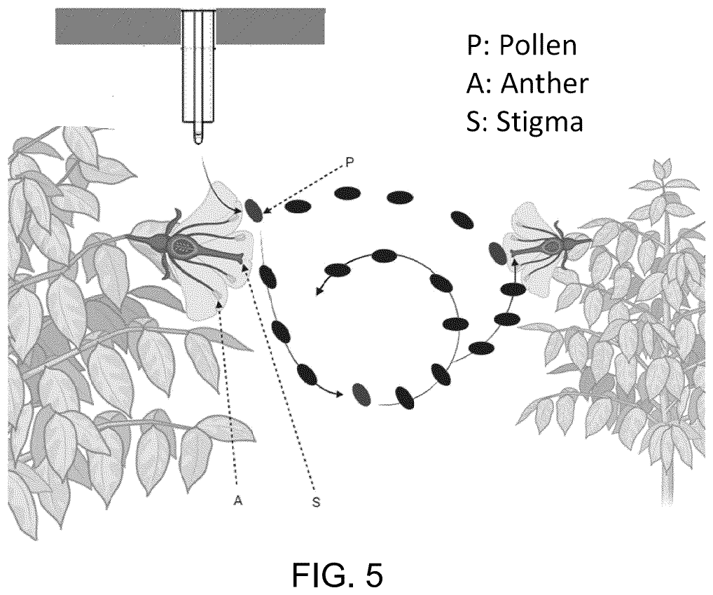

Referring now to FIG. 5, there is depicted pollen or fertigant being released from an anther and moving to a stigma of a plant by the vortices created by the positive air pressure and negative air pressure sources. There is also depicted extraneous pollen introduced to the system and said extraneous pollen moving to a stigma of a plant. As may be seen in FIG. 5, the system allows for self-pollination (pollination within the same plant) as well as cross-pollination (between plants).

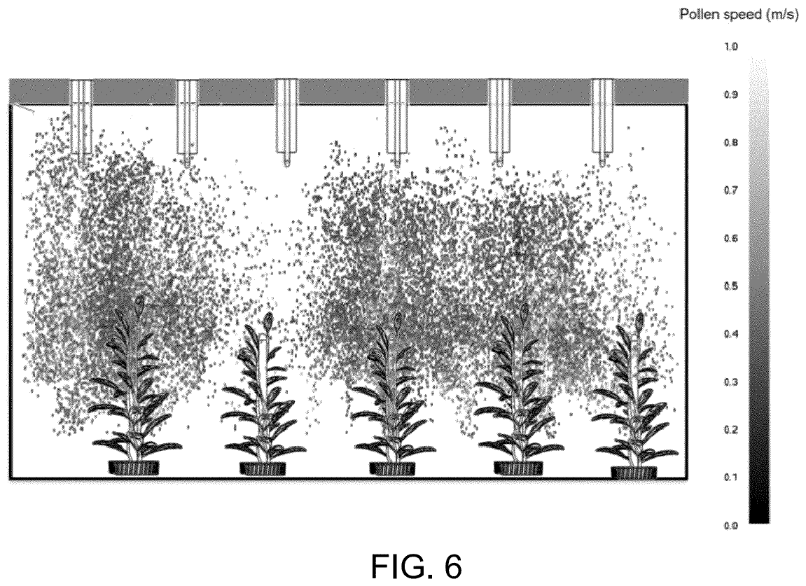

Referring now to FIG. 6, there is depicted vortices patterns of pollen introduced into the pollination system 100 as seen from the face of the platform.

Example 1—Analysis of pollen speed and distribution within the pollination system.

A model for the pollination system described herein was run wherein the distance from the top of the plant and the end of the nozzle was 365 cm and an air velocity from the end of the nozzle of 1 m/s. Air speed/pollen speed was measured at locations distant the nozzle as per the table below. As best seen in FIG. 6, the lighter depicted particles relate to a faster speed of the particles within the pollination system. It is expected that air velocities between about 0.3-0.5 m/s will achieve pollination, however it is expected that better/faster/more consistent results will be achieved at air velocity from 0.5 m/s.

The following table provides data from FIG. 6.

| Nozzle-Plant Distance | Pollen speed |
|---|---|
| Ft (cm) | m/s |
| 12 (365) | 0.3 |
| 8 (242) | 0.5 |
| 4 (120) | 1 |

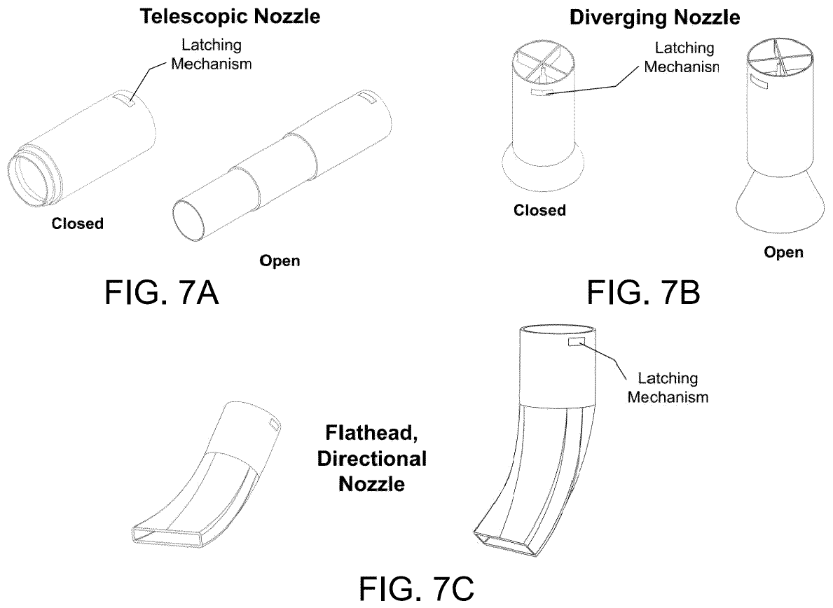

Referring now to FIG. 7a, there is depicted a telescopic adjustable nozzle in a closed or short mode and an open or elongated mode. The length adjustability facilitates the air flow as desired. Referring now to FIG. 7b, there is depicted a diverging adjustable nozzle in a closed and open mode. Referring now to FIG. 7c, there is depicted a nozzle with a direction focused end to focus flow of pollen and other ingredients to a desired location within the pollination system. Depending on the specific need, the nozzle in FIG. 7a and/or 7b and/or 7c may be selected for moving air, misting fertigants), and for moving heavier pollen.

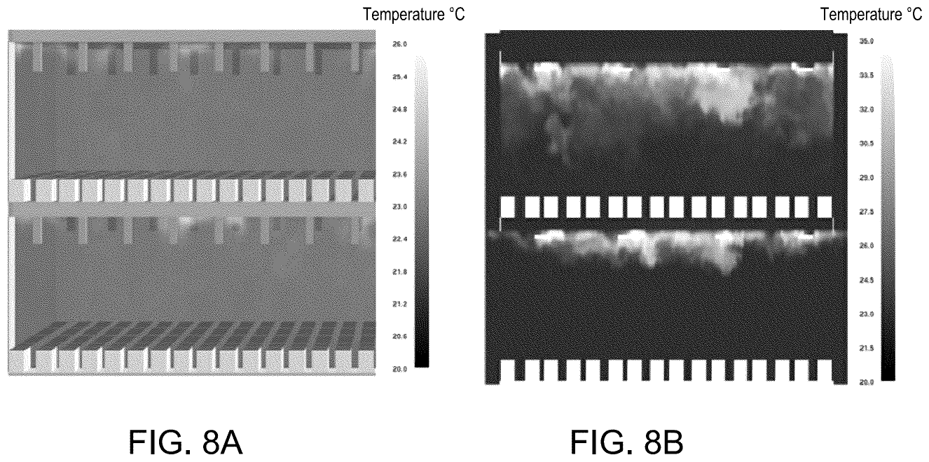

Referring now to FIG. 8A the LED light source is positioned within the air source return; wherein a substantial portion of heat generated by the LED light source located within the air source return is extracted and not introduced into the pollination system maintains a temperature controlled pollination system; FIG. 8B depicts the LED light source positioned outside the air source return, heat generated by the LED light source is dissipated to a canopy of the pollination system.

Example 2—Temperature analysis of a pollination system with a light source contained within the return air source housing versus outside the air source housing.

Two pollination systems were devised with one containing the light source within the return air source housing and one containing the light source outside the return air source housing and temperature readings were taken at various locations within the pollination system.

The following table provides data from FIG. 8A

| | | | Level#1 | | | Level#2 | | |
|---|---|---|---|---|---|---|---|---|
| | X-Coor | | Height (Z) | | Temp | Height (Z) | | Temp |
| | m | in | m | in | C. | m | in | C. |
| UGA#1 | 0.0508 | 2 | 0.3048 | 12 | 22.01 | 0.3048 | 12 | 22.05 |
| | 0.3048 | 12 | 0.3048 | 12 | 22.02 | 0.3048 | 12 | 22.02 |
| | 0.6096 | 24 | 0.3048 | 12 | 22.04 | 0.3048 | 12 | 22.02 |
| | 0.9144 | 36 | 0.3048 | 12 | 22.07 | 0.3048 | 12 | 22.02 |
| | 1.2192 | 48 | 0.3048 | 12 | 22.04 | 0.3048 | 12 | 22.01 |
| UGA#2 | 1.524 | 60 | 0.3048 | 12 | 22.03 | 0.3048 | 12 | 22.01 |
| | 1.8288 | 72 | 0.3048 | 12 | 22.02 | 0.3048 | 12 | 22.01 |
| | 2.1336 | 84 | 0.3048 | 12 | 22.02 | 0.3048 | 12 | 22.01 |
| | 2.4384 | 96 | 0.3048 | 12 | 22.01 | 0.3048 | 12 | 22 |
| UGA#1 | 0.0508 | 2 | 0.6096 | 24 | 22.01 | 0.6096 | 24 | 22.05 |
| | 0.3048 | 12 | 0.6096 | 24 | 22.03 | 0.6096 | 24 | 22.02 |
| | 0.6096 | 24 | 0.6096 | 24 | 22.04 | 0.6096 | 24 | 22.02 |
| | 0.9144 | 36 | 0.6096 | 24 | 22.10 | 0.6096 | 24 | 22.02 |
| | 1.2192 | 48 | 0.6096 | 24 | 22.03 | 0.6096 | 24 | 22.01 |
| UGA#2 | 1.524 | 60 | 0.6096 | 24 | 22.03 | 0.6096 | 24 | 22.01 |
| | 1.8288 | 72 | 0.6096 | 24 | 22.02 | 0.6096 | 24 | 22.01 |
| | 2.1336 | 84 | 0.6096 | 24 | 22.01 | 0.6096 | 24 | 22.01 |
| | 2.4384 | 96 | 0.6096 | 24 | 22.01 | 0.6096 | 24 | 22.00 |
| UGA#1 | 0.0508 | 2 | 0.9144 | 36 | 22.01 | 0.9144 | 36 | 22.04 |
| | 0.3048 | 12 | 0.9144 | 36 | 22.03 | 0.9144 | 36 | 22.02 |
| | 0.6096 | 24 | 0.9144 | 36 | 22.05 | 0.9144 | 36 | 22.02 |
| | 0.9144 | 36 | 0.9144 | 36 | 22.10 | 0.9144 | 36 | 22.02 |
| | 1.2192 | 48 | 0.9144 | 36 | 22.07 | 0.9144 | 36 | 22.01 |
| UGA#2 | 1.524 | 60 | 0.9144 | 36 | 22.04 | 0.9144 | 36 | 22.02 |
| | 1.8288 | 72 | 0.9144 | 36 | 22.03 | 0.9144 | 36 | 22.02 |
| | 2.1336 | 84 | 0.9144 | 36 | 22.03 | 0.9144 | 36 | 22.01 |
| | 2.4384 | 96 | 0.9144 | 36 | 22.03 | 0.9144 | 36 | 22.02 |
| UGA#1 | 0.0508 | 2 | 1.0922 | 43 | 22.05 | 1.2192 | 48 | 22.04 |
| | 0.3048 | 12 | 1.0922 | 43 | 22.06 | 1.2192 | 48 | 22.02 |
| | 0.6096 | 24 | 1.0922 | 43 | 22.09 | 1.2192 | 48 | 22.02 |
| | 0.9144 | 36 | 1.0922 | 43 | 22.10 | 1.2192 | 48 | 22.02 |
| | 1.2192 | 48 | 1.0922 | 43 | 22.07 | 1.2192 | 48 | 22.01 |
| UGA#2 | 1.524 | 60 | 1.0922 | 43 | 22.05 | 1.2192 | 48 | 22.02 |
| | 1.8288 | 72 | 1.0922 | 43 | 22.05 | 1.2192 | 48 | 22.02 |
| | 2.1336 | 84 | 1.0922 | 43 | 22.05 | 1.2192 | 48 | 22.02 |
| | 2.4384 | 96 | 1.0922 | 43 | 22.04 | 1.2192 | 48 | 22.02 |

The following table provides data from FIG. 8B

| | | | Level#1 | | | Level#2 | | |
|---|---|---|---|---|---|---|---|---|
| | X-Coor | | Height (Z) | | Temp | Height (Z) | | Temp |
| | m | in | m | in | C. | m | in | C. |
| UGA#1 | 0.0508 | 2 | 0.3048 | 12 | 22 | 0.3048 | 12 | 22.05 |
| | 0.3048 | 12 | 0.3048 | 12 | 22 | 0.3048 | 12 | 22.02 |
| | 0.6096 | 24 | 0.3048 | 12 | 22 | 0.3048 | 12 | 22.05 |
| | 0.9144 | 36 | 0.3048 | 12 | 22 | 0.3048 | 12 | 22.02 |
| | 1.2192 | 48 | 0.3048 | 12 | 22 | 0.3048 | 12 | 22 |
| UGA#2 | 1.524 | 60 | 0.3048 | 12 | 22 | 0.3048 | 12 | 22.12 |
| | 1.8288 | 72 | 0.3048 | 12 | 22 | 0.3048 | 12 | 22.09 |
| | 2.1336 | 84 | 0.3048 | 12 | 22 | 0.3048 | 12 | 22.10 |
| | 2.4384 | 96 | 0.3048 | 12 | 22 | 0.3048 | 12 | 22 |
| UGA#1 | 0.0508 | 2 | 0.6096 | 24 | 22 | 0.6096 | 24 | 22.71 |
| | 0.3048 | 12 | 0.6096 | 24 | 22 | 0.6096 | 24 | 22.25 |
| | 0.6096 | 24 | 0.6096 | 24 | 22.004 | 0.6096 | 24 | 22.82 |
| | 0.9144 | 36 | 0.6096 | 24 | 22.002 | 0.6096 | 24 | 22.91 |
| | 1.2192 | 48 | 0.6096 | 24 | 22.005 | 0.6096 | 24 | 22.23 |
| UGA#2 | 1.524 | 60 | 0.6096 | 24 | 22 | 0.6096 | 24 | 22.62 |
| | 1.8288 | 72 | 0.6096 | 24 | 22 | 0.6096 | 24 | 22.61 |
| | 2.1336 | 84 | 0.6096 | 24 | 22 | 0.6096 | 24 | 22.86 |
| | 2.4384 | 96 | 0.6096 | 24 | 22 | 0.6096 | 24 | 22 |
| UGA#1 | 0.0508 | 2 | 0.9144 | 36 | 22 | 0.9144 | 36 | 24.01 |
| | 0.3048 | 12 | 0.9144 | 36 | 22.6 | 0.9144 | 36 | 25.24 |
| | 0.6096 | 24 | 0.9144 | 36 | 23.0 | 0.9144 | 36 | 24.46 |
| | 0.9144 | 36 | 0.9144 | 36 | 24.8 | 0.9144 | 36 | 26.52 |
| | 1.2192 | 48 | 0.9144 | 36 | 23.3 | 0.9144 | 36 | 25.40 |
| UGA#2 | 1.524 | 60 | 0.9144 | 36 | 26.8 | 0.9144 | 36 | 28.49 |
| | 1.8288 | 72 | 0.9144 | 36 | 23.1 | 0.9144 | 36 | 24.65 |
| | 2.1336 | 84 | 0.9144 | 36 | 22.1 | 0.9144 | 36 | 23.92 |
| | 2.4384 | 96 | 0.9144 | 36 | 22.0 | 0.9144 | 36 | 22.0 |
| UGA#1 | 0.0508 | 2 | 1.0922 | 43 | 24.02 | 1.2192 | 48 | 27.25 |
| | 0.3048 | 12 | 1.0922 | 43 | 48.0 | 1.2192 | 48 | 42.83 |
| | 0.6096 | 24 | 1.0922 | 43 | 28.3 | 1.2192 | 48 | 27.08 |
| | 0.9144 | 36 | 1.0922 | 43 | 39.8 | 1.2192 | 48 | 33.42 |
| | 1.2192 | 48 | 1.0922 | 43 | 30.1 | 1.2192 | 48 | 29.39 |
| UGA#2 | 1.524 | 60 | 1.0922 | 43 | 28.1 | 1.2192 | 48 | 35.99 |
| | 1.8288 | 72 | 1.0922 | 43 | 27.2 | 1.2192 | 48 | 26.39 |
| | 2.1336 | 84 | 1.0922 | 43 | 29.8 | 1.2192 | 48 | 30.86 |
| | 2.4384 | 96 | 1.0922 | 43 | 26.3 | 1.2192 | 48 | 22.0 |

As may be seen, the temperature of the pollination system of the present disclosure FIG. 8A has minimal variance (between 22° C. and 22.1° C.) and is significantly regulated with the light source contained within the air source housing versus being contained outside the air source (between 22° C. and 48° C.) housing FIG. 8B.

As many changes can be made to the above disclosure without departing from the scope thereof; it is intended that all matter contained herein be considered illustrative and not in a limiting sense.

1. A method for pollinating at least one plant in a controlled growing environment, said at least one plant having a top, a bottom and at least one anther with exposed pollen, the method comprising:
i. applying positive air flow pressure from a top end of said controlled growing environment towards the top of said at least one plant along a first side of said at least one plant towards said bottom of said at least one plant, wherein said first side of said at least one plant is proximate an outside perimeter of said controlled growing environment; and
ii. applying negative air flow pressure resulting in drawing air flow from said bottom of said at least one plant upwards along a second side of said at least one plant towards said top of said at least one plant towards said top of said top end of said controlled growing environment, wherein said second side of said at least one plant is distant said outside perimeter of said controlled growing environment;
whereby the positive air flow pressure has an air flow speed such that along with said negative air flow pressure generate a turbulent air flow vortex creating i) an air curtain along the outside perimeter of said controlled growing environment and ii) promoting releasing and mixing of pollen from the at least one anther with exposed pollen and iii) circulating the pollen from the at least one anther with exposed pollen to at least one stigma of the at least one plant in a vicinity proximal and distal the at least one plant resulting in pollination.

2. The method of claim 1 further comprising controlling temperature and moisture of air supplied by the positive air flow pressure.

3. The method of claim 1 further comprising fertilizing the plant through the positive air flow pressure.

4. The method of claim 1 further comprising misting the plant through the positive air flow pressure.

5. The method of claim 1 further comprising fertigating the plant through the positive air flow pressure.

6. The method of claim 1 further comprising collecting pollen released from the at least one anther by the negative air flow pressure.

7. The method of claim 1 further comprising introducing extraneous pollen to the controlled growing environment.

8. The method of claim 1 further comprising providing light to the controlled growing environment, wherein heat from said light is contained by the negative air flow pressure.

9. A method for pollinating at least one plant in a controlled growing environment having at least two spaced apart plants, each of said at least two spaced apart plants having a top, a bottom and at least one anther with exposed pollen and at least one stigma, the method comprising:
i. applying positive air flow pressure from a top end of said controlled growing environment towards the top of said at least two spaced apart plants along a first side of one of said at least two spaced apart plants towards said bottom of one of said at least two spaced apart plants, wherein said first side of one of said at least two spaced apart plants is proximate an outside perimeter of said controlled growing environment; and
ii. applying negative air flow pressure resulting in drawing air flow from said bottom of said at least two spaced apart plants upwards along a second side of another of said at least two spaced apart plants towards said top end of said controlled growing environment, wherein said second side of said another of said at least two spaced apart plants is distant said outside perimeter of said controlled growing environment;
whereby the positive air flow pressure has an air flow speed such that along with said negative air flow pressure generate a turbulent air flow vortex creating i) an air curtain along the outside perimeter of said controlled growing environment and ii) promoting releasing and mixing of the pollen from the at least one anther with exposed pollen and iii) circulating the pollen from the at least one anther with exposed pollen to at least one stigma of the another of said at least two spaced apart plants resulting in pollination.

10. A pollination system for a controlled growing environment, comprising:
i. at least one support; a top, a bottom, a front, a back, a top central portion, an outside perimeter and two sides;
ii. at least one plant, preferably at least two plants, most preferably a plurality of plants, said at least one plant having a top and a bottom and at least one anther with exposed pollen and at least one stigma; said at least one plant supported by the at least one support;
iii. at least one air source proximate a side of said top of said controlled growing environment for positive air flow pressure from the top of said at least one plant along a first side of said at least one plant towards said bottom of said at least one plant, wherein said at least one air source is offset from a center of said at least one plant;
iv. at least one air source return proximate said top central portion of said controlled growing environment applying negative air flow pressure from the bottom of said at least one plant along a second side of said at least one plant towards said top of said at least one plant, wherein said second side of said at least one plant is distant said outside perimeter of said controlled growing environment; whereby the positive air flow pressure and the negative air flow pressure generate a turbulent air flow vortex, preferably a plurality of turbulent air flow vortices, creating i) an air curtain along the outside perimeter of said controlled growing environment and ii) promoting releasing and mixing of the pollen from the at least one anther with exposed pollen and iii) circulating the pollen from the at least one anther with exposed pollen to at least one stigma of the at least one plant, in a vicinity proximal and distal the at least one plant, resulting in pollination; and
v. at least one light source integrated with said at least one air source return.

11. The pollination system of claim 10 further comprising at least one of a:
i. fertilizer source;
ii. misting source;
iii. fertigation source; and/or
iv. combinations thereof.

12. The pollination system of claim 10 further comprising a pollen collector.

13. The pollination system of claim 12 wherein said pollen collector is said at least one air source return.

14. The pollination system of claim 10 further comprising an extraneous pollination source.

15. The pollination system of claim 14 wherein extraneous pollination is delivered by said at least one air source applying positive air flow pressure along said first side of said at least one plant.

16. The pollination system of claim 10 wherein the at least one air source further comprises a temperature and humidity controller.

17. The pollination system of claim 10 wherein the at least one air source comprises an adjustable nozzle adjustable in at least one of length, direction, flow rate and combinations thereof.

18. The pollination system of claim 10 wherein the at least one air source is positioned 500 centimetres above said top of said plant, preferably between 0-365 centimetres above said top of said plant, more preferably between 1-200 cm above said top of said plant, and an air speed of from about 1-0.5 m/s, more preferably from 0.3-0.5 m/s, respectively.

19. The pollination system of claim 10 wherein the at least one air source return is positioned 500 centimetres above said top of said plant, preferably between 0-365 centimetres above said top of said plant, more preferably between 1-200 cm above said top of said plant, and an air speed from about 1-0.5 m/s, more preferably from 0.3-0.5 m/s, respectively.

20. The pollination system of claim 10 wherein the at least one air source is offset to one side of said plant.
